(19) United States
(12) Patent Application Publication
(10) Pub. No.: US 2016/0120712 A1
(43) Pub. Date: May 5, 2016

(54) TESTOSTERONE KIT

(71) Applicant: ASCLEMED USA, INC. dba EnovaChem Manufacturing, Torrance, CA (US)

(72) Inventor: Robert P. Nickell, Torrance, CA (US)

(21) Appl. No.: 14/925,848

(22) Filed: Oct. 28, 2015

Related U.S. Application Data
(60) Provisional application No. 62/072,298, filed on Oct. 29, 2014.

(51) Int. Cl. A61F 17/00; A61K 9/00; A61K 31/568

(52) U.S. Cl. CPC A61F 17/00; A61K 31/568; A61K 9/0019

(57) ABSTRACT

Provided a kit with all the necessary components for a medical practitioner in the office practice setting to inject testosterone to a patient. Specifically, the kit can include a holder and following components placed inside a holder: a) a container of testosterone ester in a formulation that is suitable for injection to an animal; b) a pad for disinfecting or cleaning an injection site; and c) a pair of gloves.

### CROSS-REFERENCE

The present application claims the benefit of U.S. provisional application No. 62/072,298, filed on Oct. 29, 2014, which is incorporated herein by reference in its entirety.

### BACKGROUND SECTION OF THE INVENTION

With the changing reimbursements, types of health care insurance, and new regulations with regards to treatment, a medical practitioner and his/her team when treating a patient is under immense time pressures to perform quickly, efficiently and with safety for the patient. If a patient requires a singular or even multiple injections, the medical practitioner must rely on his/her team to first locate the necessary and correct injectable medications, insure they are the correct strength and percentage, insure dating is current, insure the top has not been contaminated, insure the liquid is not crystallized, or containing growth, insure there is sufficient medication in the vial, and insure that the injection has been stored at the proper temperature and humidity as well as all other possible, related materials and safety precautions needed to treat the patient. The medical practitioner can lose valuable time trying to not only locate the necessary drugs and materials to treat the patient but as well to insure the compliance, the safety of the patient by preventing contamination. In some instances, not all the drugs and materials may be readily available, in the current exam room, resulting in delayed treatment and possible and potential medical errors. In addition, the medical practitioner or his/her team may make a mistake by incorrectly choosing the drug, by incorrectly identifying the product, not checking the expiration date, not returning it to the proper environment for storage, or not having enough product and thus requiring an additional sterile vial to be opened to inject directly into the veins of a patient.

There is a need in the art for a convenience kit that solves the above mentioned problems in the medical practitioner office.

### SUMMARY SECTION OF THE INVENTION

Provided is a kit comprising a holder and following components placed inside a holder: a) a container of testosterone ester; b) a pad for disinfecting or cleaning an injection site; c) a pair of gloves; and d) optionally a CSR wrap. The testosterone ester can be testosterone cypionate or testosterone enanthate. The strength of the testosterone ester can be about 25 mg/ml to about 250 mg/ml, such as 100 mg/ml or 200 mg/ml. The amount of the testosterone ester can be about 1 to about 10 ml, such as about 1 ml, about 5 ml or about 10 ml. In one embodiment, testosterone cypionate at a strength of 200 mg/ml and amount of 1 ml is used. The pad can be an alcohol pad, such as an isopropyl pad. The isopropyl pad can have a strength of about 70%. The kit can further include an instruction sheet.

Provided is a kit comprising a box with a lit and following components placed inside a holder: a) a container of testosterone cypionate with a strength and an amount of 200 mg/ml (1 ml) USP (United States Pharmacopeia) in a formulation that is suitable for injection to an animal; b) an alcohol pad for disinfecting or cleaning an injection site; c) a pair of gloves; d) a CSR wrap; and e) an instruction sheet.

### BRIEF DESCRIPTION OF THE FIGURES

FIG. 1 illustrates a box for placing the various components of the kit.

FIG. 2 illustrates an instruction sheet.

FIG. 3 illustrates a box for placing a drug vial.

FIG. 4 illustrates a drug vial.

FIG. 5 illustrates a drug ampule.

FIG. 6 illustrates a CSR wrap.

FIG. 7 illustrates a glove.

FIG. 8 illustrates a pad.

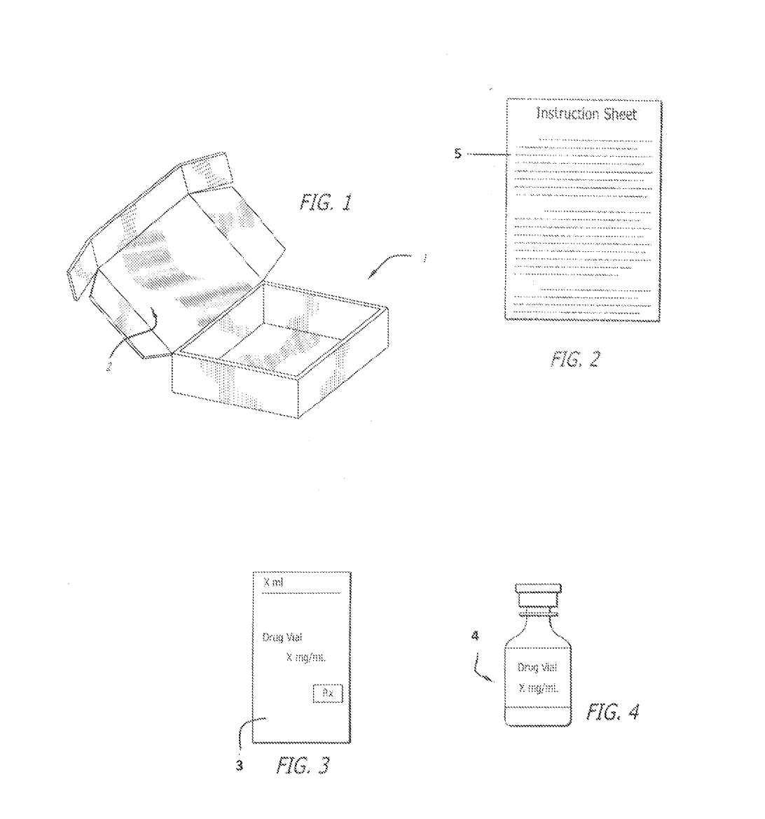

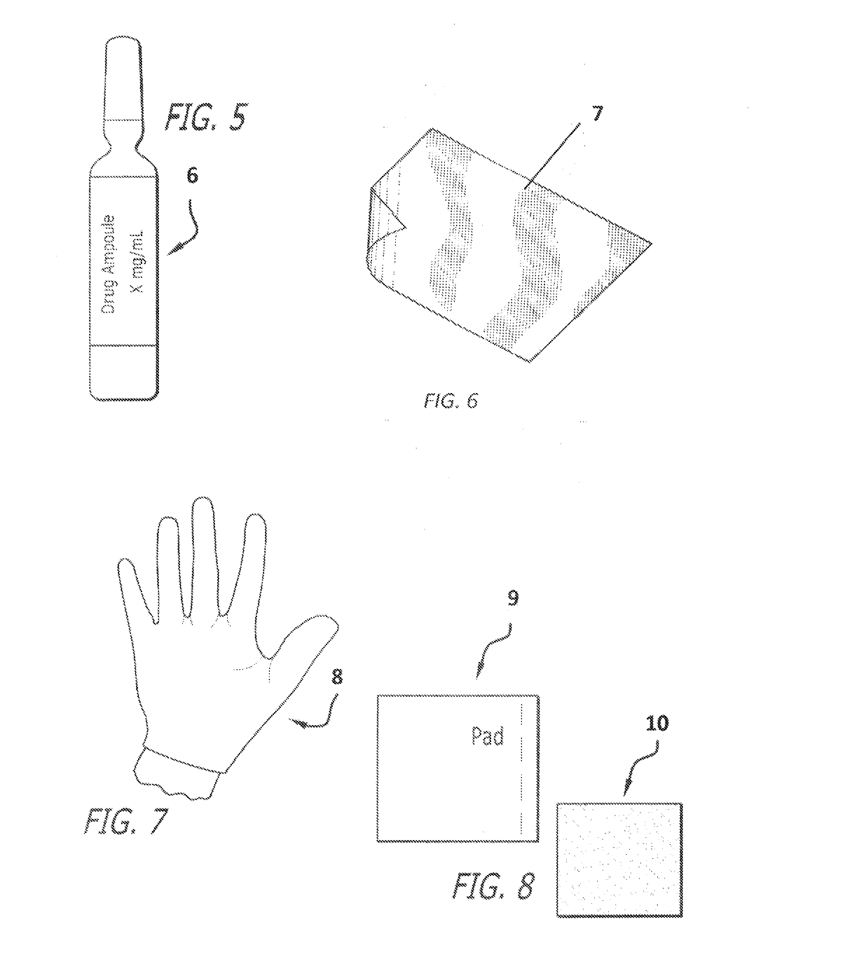

### DETAILED DESCRIPTION OF THE INVENTION

The present invention provides a kit with all the necessary components for a medical practitioner in the office practice setting to inject testosterone to a patient.

The components of the kit can be put in a holder, such as a box (1). The box (1) can be a rectangular cube which has a; peel-off lid (2) on top for opening the box.

One component of the kit can be testosterone ester for injection. The testosterone ester can be testosterone cypionate or testosterone enanthate. The strength of the testosterone ester can be about 25 mg/ml to about 250 mg/ml, such as about 100 mg/ml or about 200 mg/ml. The amount of the testosterone can be about 1 to about 10 ml, such as about 1 ml, 5 ml and 10 ml. The testosterone ester can be dissolved in an oil, such as grape seed oil, peanut oil, cottonseed oil, or sesame oil. A 200 mg/ml solution of testosterone cypionate can include 200 mg of testosterone cypionate USP, 0.2 ml of Benzyl Benzoate, USP, 560 mg of Cottonseed Oil, USP, and 9.45 mg of Benzyl Alcohol, USP. In another formulation, each mL of the injectable solution provides 200 mg testosterone enanthate. USP in sesame Oil, USP with 5 mg chlorobutanol, USP (chloral derivative) as a preservative. In one embodiment, testosterone cypionate at a strength of 200 mg/ml (1 ml) is used. A container (ampoule (6) or vial (4)) of testosterone cypionate in a form (such as a suspension or solution) that is ready for injection comes with the kit.

One component of the medical kit can be a pair of sterile gloves (8). The glove (8) can be any suitable glove that is used by a medical practitioner, such as latex or nitrile gloves. In one embodiment, a pair of nitrile gloves are part of the kit. Nitrile gloves are a type of disposable glove made of Nitrile rubber, also known as Buna-N, Perbunan, or NBR. Nitrile gloves contain no latex. A medical practitioner can tear a package containing the gloves (8) and wear the gloves before performing an injection to help prevent contamination and increase compliance.

One component of the kit can be a CSR (Central Supply Room) wrap (7). The CSR wrap can be put on a tray or a surface before placement of the other components of the kit. The CSR wrap keeps a clean environment.

One component of the medical kit can be alcohol swabs (pads) (10), which is an antiseptic for preparation of the skin prior to injection. The alcohol swabs can be individually foil wrapped (9). The alcohol swabs can contain 70% or another amount of isopropyl alcohol.

In one embodiment needles and syringes are not included.

The kit can contain an instruction sheet (5). The instruction sheet can identify one or more components of the kit, provide instructions as to how to use each component, and optionally suggest an order of use for the components of the kit.

The kit can be used to inject a male with testosterone as part of replacement therapy in the male in conditions associated with symptoms of deficiency or absence of endogenous testosterone. The kit can be used in humans, dogs, horses, or other mammalian animals.

When a medical practitioner decides to carry out the injection, the medical practitioner opens the box, puts on the gloves, opens the CSR wrap and puts the CSR wrap on a surface. The medical practitioner then disinfects the injection site with the isopropyl pad and injects the testosterone with the use of, a syringe that is not provided in the kit. After the injection, the medical practitioner discards the kit and its component. The medical practitioner may also read the instruction sheet.

The following table provides an example of the components of the kit:

TABLE 1
1 Testosterone Cypionate Injection. USP 200 mg/ml (1 mil) (distributed by
Perrigo)
Each mL contains Testosterone Cypionate, 200 mg; Benzyl Benzoate,
0.2 ml; Benzyl Alcohol, 9.45 mg added as a preservative; cottonseed oil,
560 mg.
1 Isopropyl Alcohol 70% Prep Pad (Dynarex Corporation)
Each pad is saturated with 70% isopropyl alcohol
1 Pair Nitrile Powder Free Sterile Gloves (Non-Latex)• (Size 7.5)
1 CSR Wrap

What is claimed is:

1. A kit comprising a holder and following components placed inside a holder: a) a container of testosterone ester in a formulation that is suitable for injection to an animal; b) a pad for disinfecting or cleaning an injection site; and c) a pair of gloves.

2. The kit of claim 1, further comprising a CSR wrap.

3. The kit of claim 1, wherein the testosterone ester is testosterone cypionate or testosterone enanthate.

4. The kit of claim 1, wherein the strength of the testosterone ester is about 25 mg/ml to about 250 mg/ml USP (United States Pharmacopeia).

5. The kit of claim 1, wherein amount of the testosterone ester is about 1 to about 10 ml.

6. The kit of claim 1, wherein the testosterone ester is testosterone cypionate with a strength of 200 mg/ml USP (United States Pharmacopeia) and an amount of 1 ml.

7. The kit of claim 1, wherein the pad is an alcohol pad.

8. The kit if claim 7, wherein the pad is an isopropyl pad.

9. The kit of claim 1, further comprising an instruction sheet.

10. The kit of claim 1, wherein a needle or a syringe is not included.

11. A kit comprising a box with a lit and following components placed inside a holder: a) container of testosterone cypionate with a strength and an amount of 200 mg/ml (1 ml) USP (United States Pharmacopeia) in a formulation that is suitable for injection to an animal; b) an alcohol pad for disinfecting or cleaning an injection site; c) a pair of gloves; d) a CSR wrap; and e) an instruction sheet.
